(19) United States
(12) Patent Application Publication
(10) Pub. No.: US 2012/0283547 A1
(43) Pub. Date: Nov. 8, 2012

(54) MAGNETIC RESONANCE IMAGING USING VELOCITY SELECTIVE EXCITATION

(75) Inventors: Eric C. Wong, Del Mar, CA (US); Jia Guo, La Jolla, CA (US)

(73) Assignee: THE REGENTS OF THE UNIVERSITY OF CALIFORNIA, Oakland, CA (US)

(21) Appl. No.: 13/444,791

(22) Filed: Apr. 11, 2012

Related U.S. Application Data
(60) Provisional application No. 61/474,181, filed on Apr. 11, 2011.

(51) Int. Cl. A61B 5/055

(52) U.S. Cl. 600/410

(57) ABSTRACT

Techniques and systems are disclosed for magnetic resonance imaging by applying velocity-selective excitation. In some implementations, velocity selective excitation pulses are applied to a subject, causing an observable magnetization in the subject. An image of the subject is generated based on the observable magnetization. In some implementations, additionally, velocity selective excitation is combined with arterial nulling pulses such that the contribution from the arterial portion of a subject is excluded from the observable magnetization in the subject.

### CROSS REFERENCE TO RELATED APPLICATIONS

The present application claims the benefit of priority from the U.S. Provisional Patent Application 61/474,181, filed on Apr. 11, 2011. The entire content of the before-mentioned patent application is incorporated by reference as part of the disclosure of this application.

### STATEMENT REGARDING FEDERAL RIGHTS

This invention was made with government support under grant no. EB002096 awarded by the National Institute of Health (NIH). The government has certain rights in the invention.

### BACKGROUND

This application relates to devices and techniques for magnetic resonance imaging (MRI).

Imaging through MRI techniques is well known and has been widely applied in imaging applications in medical, biological and other fields. In essence, a typical MRI technique produces an image of a selected body part of an object under examination by manipulating the magnetic spins in a body part and processing measured responses from the magnetic spins. MRI systems may include hardware to generate different magnetic fields for imaging, including a static magnetic field along a z-direction to polarize the magnetic spins, gradient fields along mutually orthogonal x, y, or z directions to spatially select a body part for imaging, and an RF magnetic field to manipulate the spins.

Improvements in MRI techniques are needed.

### SUMMARY

Techniques and structures and apparatus are disclosed to use velocity selective excitation in obtaining magnetic resonance images of a subject. In one aspect, the disclosed techniques are useful in mapping venous oxygenation.

The subject matter described in this specification potentially can provide one or more of the following advantages. The described techniques, apparatus and systems can be used for MR mapping of Oxygenation/OEF (Oxygen Extraction Fraction) for identification of penumbra and monitoring of penumbra during and/or after treatment. Also, the described techniques, apparatus and systems can be used for MR mapping of Oxygenation/OEF for evaluation of tumors. Additionally, the described techniques, apparatus and systems can be used for MR mapping of Oxygenation/OEF for functional studies.

In addition, venous oxygenation can be mapped non-invasively, using MRI, without the use of contrast agents. The described techniques, systems and apparatus can provide a way of specifically exciting moving spins, which allows for separation of blood signal from tissue signal without subtraction, resulting in higher time efficiency than existing methods. Also, proximal inversion pulses can be incorporated to null the arterial blood signal at the time of image acquisition, allowing for the separation of venous blood signal from arterial blood signal, and accurate measurement of venous T2 and oxygenation. At the same time, the static tissue water that gives rise to the venous signal has more time to relax and provide higher venous blood signal, thus increasing SNR. Additionally, multi-echo spin echo acquisition provides a faster measurement of venous T2 than T2 preparation, and thus higher time efficiency.

Other advantages can include: 1) higher SNR due to more relaxed venous blood; 2) no subtraction gives higher time efficiency; 3) insensitive to physiological noise; 4) a T2 map is generated every TR from multi-echo acquisition.

### BRIEF DESCRIPTION OF THE DRAWINGS

FIGS. 1A and 1B show Mz evolution in QUIXOTIC (Quantitative Imaging of Extraction of Oxygen and Tissue Consumption) and in the described VSEAN method with only the signal from spins that will be imaged. Venous signal in QUIXOTIC is 0.47 in every two TRs, compared to 0.86 in VSEAN in only one TR.

FIG. 2 shows a pulse sequence diagram.

FIG. 3A shows a reference image.

FIG. 3B shows acquired data.

FIG. 3C shows a venous blood signal after projection.

FIG. 3D shows an estimated relative venous blood volume.

FIG. 3E shows a T2 map.

FIG. 3F shows a histogram of T2 values in FIG. 3E.

FIG. 4 shows a Velocity Selective BIR-4 pulse.

FIG. 5 shows a simulated response of VS-BIR-4 pulse across velocity and B₁.

FIG. 6 shows a comparison of VS-BIR-4 and double sech pulse.

FIG. 7 shows VS-ASL images using BIR-4 (top) and double sech (bottom) pulses.

FIG. 8 shows exemplary T2 measurement in a gel phantom using only a T2 preparation pulse.

FIG. 9 shows an exemplary signal acquired from a stationary gel phantom using a VSE pulse without (a) and with (b) eddy current compensation, expressed as a fraction of the unsuppressed signal.

FIG. 10 shows an example provided by using higher cutoff velocity of 3 cm/s instead of the original 2 cm/s; the signal from the blood is clearly matched with the vessel structures, and an effective suppression of static tissue signal is observed.

FIG. 11 shows characteristics of separate venous blood, arterial blood and static tissue (including CSF).

FIG. 12 shows exemplary signals used in subtracting contribution of arterial signals.

FIG. 13 shows relationship between magnetization spins and velocity-selective excitation pulses.

FIG. 14 shows the effect of velocity-selective excitation pulses on magnetization spins caused in a subject.

FIG. 15 show an exemplary process and associated signals for implementing VSEAN.

FIG. 16 shows a block diagram of an exemplary data processing technique.

FIG. 17 shows tissue suppression on a silicone phantom.

FIGS. 18-22 show comparisons of VSEAN and QUIXOTIC.

FIG. 23 shows a comparison of the venous signal between VSEAN and QUIXOTIC.

FIG. 24 is a table shows a summary of comparison between VSEAN and QUIXOTIC.

FIG. 25 is a flow chart representation of a procedure of magnetic resonance imaging.

FIG. 26 is a block diagram representation of an MRI apparatus.

Like reference symbols and designations in the various drawings indicate like elements.

### DETAILED DESCRIPTION

In the description below, section headings are used only for improving readability of the disclosed subject matter and do not limit the scope of the disclosed subject matter in any way. Furthermore, while the techniques are disclosed with specific references to magnetic resonance imaging, the usefulness of these techniques to other medical applications such as medical resonance angiography will be appreciated by one of skill in the art.

Magnetic Resonance Imaging (MRI) is an important medical diagnostic tool, allowing a medical practitioner to obtain information about internals of a subject (e.g., a patient) in a non-invasive manner. In a typical MRI procedure, a magnetic field (e.g., electromagnetic pulses) is applied to the subject and results of magnetization are obtained using sensors (e.g. coils). The data thus obtained is processed to produce images of the subject.

One operational challenge faced by present MRI systems is that the magnetic field typically induced in the subject is along a longitudinal axis, which makes the observation and measurements of this field hard in practice. Furthermore, often, the subject under MRI is an organ that includes a large portion of tissue (e.g., 97% tissue, which typically is static) and a small portion of blood (e.g., 3% blood, which is typically moving). In many medical applications, a medical practitioner is more interested in the MRI readings obtained from the blood, and not from the tissue. The present day systems sometimes find it hard to filter out the contribution to an MRI image from tissue, while leaving the contribution from blood unaltered.

In addition, often, medical practitioners need to find out quantities such as venous oxygenation that are diagnostically useful. Even when the contribution from tissue is eliminated from an MRI, it is still desirable to subtract the contribution from arterial flows, which makes the observation of venous oxygenation simpler and more accurate.

The techniques, systems and apparatus described in this patent document are useful to overcome the above-discussed limitations of present MRI systems, in addition to other benefits discussed herein. In one aspect, the disclosed techniques enable imaging of a subject patient by excluding contribution from static portion of the subject. In another aspect, the use of an arterial nulling technique enables subtraction of arterial blood contribution to the resulting MRI image.

The techniques, systems and apparatus described in this application can be used in such medical applications where imaging of moving parts is useful, e.g., to map venous oxygenation. In one aspect, described is a way of mapping venous oxygenation non-invasively, using MRI, without the use of contrast agents. The described techniques, systems and apparatus can provide a way of specifically exciting moving spins, which allows for separation of blood signal from tissue signal without subtraction, resulting in higher time efficiency than existing methods. Also, proximal inversion pulses can be incorporated to null the arterial blood signal at the time of image acquisition, allowing for the separation of venous blood signal from arterial blood signal, and accurate measurement of venous T2 and oxygenation. At the same time, the static tissue water that gives rise to the venous signal has more time to relax and provides higher venous blood signal, thus increasing the signal-to-noise ratio (SNR) of the measurement. Additionally, multi-echo spin echo acquisition provides a faster measurement of venous T2 than T2 preparation, and thus higher time efficiency.

The techniques can include (1) applying radiofrequency and gradient pulses to invert arterial blood magnetization supplying to the tissue of interest. Also (2) time on the order of hundreds of milliseconds is allowed to pass in order for the inverted arterial magnetization to enter the imaging plane, decelerate and replace non-inverted arterial magnetization. In addition, (3) around the null point of the arterial blood, (3.1) a few images of stationary magnetization mainly from tissue is collected to give the phase of the tissue signal without using velocity-selective excitation; and (3.2) Velocity-Selective pulses which creates Velocity-Selective Excitation are applied to collect only the venous blood signal.

Also, (4) multi-echo spin echo acquisition is used to collect T2-weighted images at different echo times. Because flow can affect even and odd echoes differently, accurate T2 measurements can be made using a combination of: 1) using only even echoes; 2) using flow compensation; and 3) using adiabatic non-selective refocusing pulses. In some implementations (4.1), T2 preparation can be used, as in (1 and 2) above, which would work, but with lower time efficiency.

In some implementations, the above steps may be repeated several times. Additionally, stationary signal is reduced further by using the reference phase collected in step 3.1 in data processing. A venous T2 map is calculate using curve fitting.

Imaging of Oxygen Extraction Fraction Using Velocity Selective Excitation with Arterial Nulling (VSEAN)

Introduction:

Oxygen Extraction Fraction (OEF) is thought to be a more specific indicator of ischemic penumbra in stroke than contrast based perfusion MRI. The Cerebral Metabolic Rate of Oxygen (CMRO₂), which can be calculated from OEF and Cerebral Blood Flow (CBF) provides a quantitative measure of brain metabolism, and can be used to study brain physiology and function. Such usefulness of OEF or CBF provides a strong motivation for robust, non-invasive measures of OEF. Bolar et at introduced a new imaging method to differentiate the post-capillary venular (PCV) blood from other tissues using velocity-selective (VS) pulses, and used T2 measurements in the PCV blood to estimate OEF. Here we introduce a strategy to image the PCV blood with higher signal to noise ratio (SNR) using VS Excitation with Arterial Nulling (VSEAN).

Theory:

The source of signal in brain can be classified into static and moving spins, the latter consisting primarily of arterial and venous blood. Since venous blood is the target in OEF experiments, contribution to the measured signal from static tissue and arterial blood should be removed. In the method described below in detail, a slab selective inversion pulse is applied just below the imaging plane as in a typical Pulse Arterial Spin Labeling (PASL) experiment, and the inversion time is chosen so that arterial blood is nulled during image acquisition. To remove the static tissue signal, a modified Velocity-Selective BIR4 pulse train with Mz response proportional to sin(v) (VS-sin) is applied. For cutoff velocity v_{cut}, the longitudinal magnetization can be expressed as: Mz(v)=sin(πv_{cut}), note that the spins with positive and negative velocities have opposite phases. A second VS-sin module can be applied to create a sin² modulation: Mz(v)=sin²(πv_{cut}). Because of this symmetric and quadratic response near zero velocity, static and slow moving spins are well suppressed. We use as a second VS-sin module simple diffusion gradients in the imaging sequence to shorten the preparation time and most importantly, to keep the flowing spins out of phase with residual static tissue signal, allowing for additional phase sensitive suppression of static tissue.

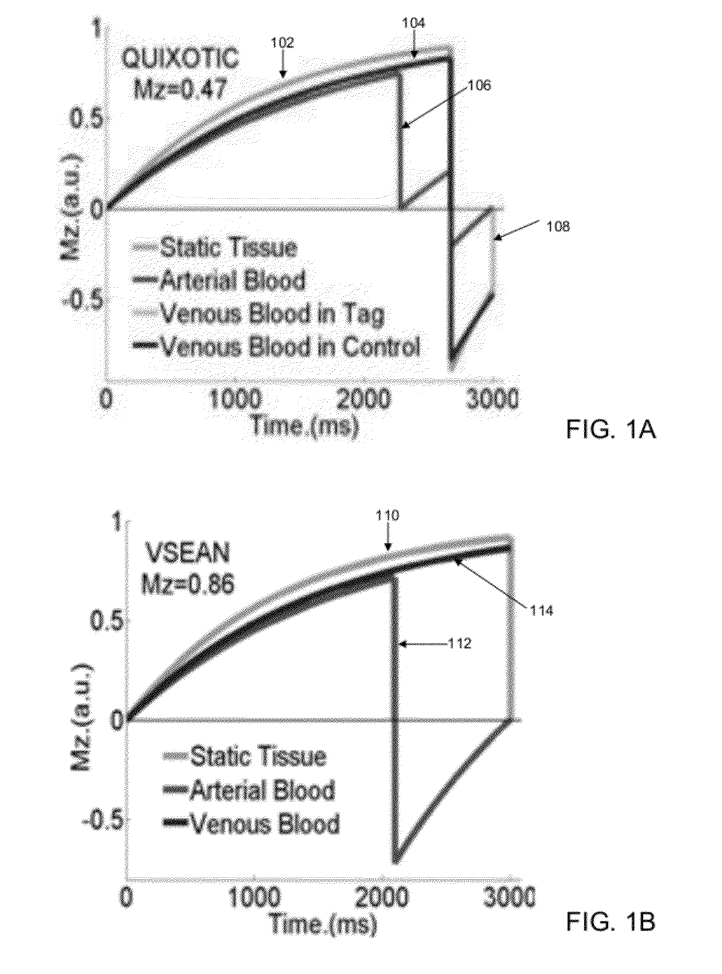

With reference to FIGS. 1A and 1B, results obtained using the above discussed method are compared with results obtained using QUIXOTIC method for obtaining MRI images. QUIXOTIC subtracts out static tissue signal, and filters venous blood signal based on deceleration. However, after the first VS module, an in-plane inversion pulse is applied in QUIXOTIC to null the partially recovered arterial blood signal. This pulse also inverts venous blood, and the signal of venous blood is reduced due to inversion recovery (FIGS. 1A and 1B). In FIG. 1A, curve 102 depicts resulting contribution of static tissue, curve 104 represents the contribution by arterial blood, curve 106 represents contribution by venous blood in tag and 108 represents contribution by venous blood in control. In FIG. 1B, curve 110 represents contribution by static tissue, curve 112 represents contribution by arterial blood and curve 114 represents contribution by venous blood.

The magnetization of venous blood with VSEAN is 0.86, compared to 0.47 in QUIXOTIC with TR=3 s, T1ₐᵣₜₑᵣᵢₐₗ=1664 ms, T1ᵥₑₙₒᵤₛ=1500 ms, T1ₜᵢₛₛᵤₑ=1200 ms. Since no subtraction is used in VSEAN, another factor of improvement of SNR per unit time is achieved. Because the static tissue signal is suppressed in VSEAN, VSEAN may be less sensitive to physiological noise. Assuming that venous spins are distributed across a large velocity range, up to half of the signal is lost in VSEAN is due to the sin²(πv_{cut}) modulation. However, the overall SNR improvement per unit time is still 1.29 times that of QUIXOTIC. The measurement of oxygenation of venous blood can either be carried out with T₂ prep or multiple spin echo acquisition.

Pulse Sequence and Methods:

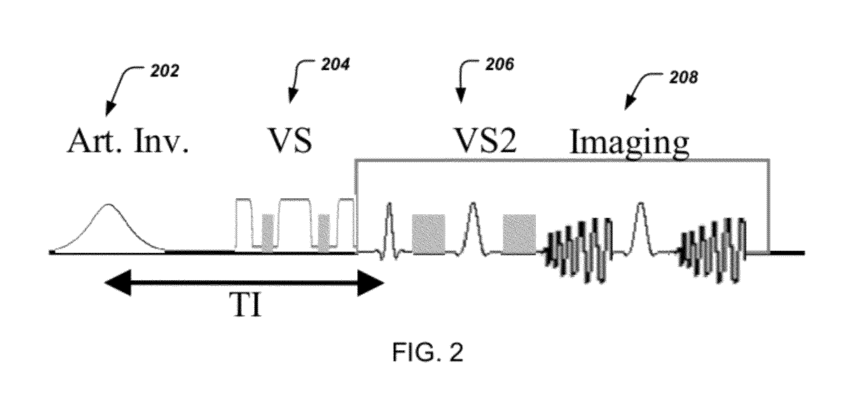

FIG. 2 depicts one example of a pulse sequence 200 applied to achieve VSEAN MRI. As shown in FIG. 2, a proximal sech pulse 202 is applied at TI to invert arterial blood magnetization spins. The first (velocity selective (VS) module 204 is applied right before image acquisition, the second VS module 206 is incorporated into the image acquisition, and multiple spin echoes 208 are collected with spiral readout. To collect reference images, the first VS pulse train 204 has cos(πv_{cut}) modulation, while in actual data acquisition, VS-sin modulation is applied (206). The diffusion gradients in the imaging part remain the same for reference and data collection.

In one implementation, a young male subject was scanned on a GE 3T system with 8-channel head coil. The imaging parameters were: single slice with FOV=220 mm*8 mm, TR=4 s, TE=21/41/61 ms, TI=1013 ms, v_{cut}=2 cm/s, gradients along S/I direction, inversion band=15 cm, 10 mm gap between inversion and imaging region, spatial spectral excitation with sinc 180° CPMG refocusing pulses, spiral read out with matrix size 32, interpolated to 64; 80 images plus 4 reference images (total 84 scans) were collected.

Data Processing and Results:

The data from each coil was complex-reconstructed individually in order to preserve the phase information, then spatially smoothed by a 3*3 Gaussian kernel and projected onto the axis perpendicular to the phase estimated from reference image with the same TE. The projected signal from coils were combined using the sensitivity map for coil, I, estimated also from the reference images:

$${S}_{comb} = {\sum }_{i} {S}_{idata} {Sen}_{i} ( e . g . , {s}_{comb} = {\sum }_{j} {s}_{jdata} {sen}_{i} ) , \\ where {sen}_{i} = \frac{\vert {S}_{iTEi} \vert}{{\sum }_{i} \vert {S}_{iTEi} \vert} ( e . g . , {sen}_{i} = \frac{\vert \text{?} \vert}{\text{?}} ) . \\ \text{?} \text{indicates text missing or illegible when filed} \qquad Equation ( 1 )$$

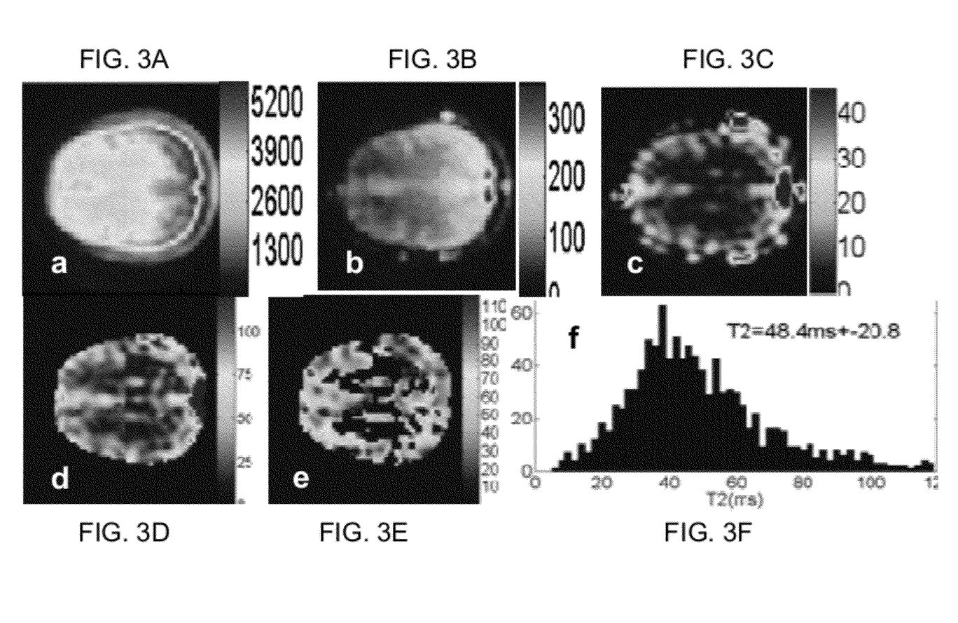

With this type of signal combination, the noise has zero mean and does not bias the T2 estimates. A T2 map was calculated using echoes 1 and 3 because of the even echo rephasing phenomenon. A threshold from the relative venous blood volume map (FIG. 3D) after T2 calculation masked out the regions in which mean T2 value and its standard deviation are estimated.

As shown in FIG. 3B, the VS-sin module suppressed the static to <5%, and after projection (FIG. 3C) the static tissue signal was below 0.1%, leaving venous blood signal (since arterial blood was nulled at this acquisition time). The calculated venous T2 values were 48.4±20.8 ms, giving a venous oxygen saturation of 58% using a T2/Yv calibration curve, and an OEF of 42% assuming fully oxygenated arterial blood. Note that the high T2 values in FIG. 3E were at the low relative blood volume region, which may be contaminated by noise.

Discussion:

Other strategies could be considered to further reduce the static signal: 1) alternate the sign of the moving spin signal every TR without affecting the static tissue signal, either by adding an extra 0/π phase shift to the VS-sin module, or by changing the sign of the diffusion gradients in the imaging sequence; 2) apply a tip-down pulse right before the readout gradients to tip the static tissue signal onto longitudinal axis.

BIR-4 Based B1 and B0 Insensitive Velocity Selective Pulse Trains

Introduction:

The B1 Insensitive Rotation (BIR-4) pulse was recently shown to be useful for B1 insensitive T2 preparation. In some implementations, two equal delays can be added to the BIR-4 pulse at the zero points of the RF amplitude, and the resulting pulse was demonstrated to be both B₁ and Bₒ insensitive. In some implementations, the BIR-4 pulsing technique can be modified to use symmetrical gradient pulses during the delay periods to impart velocity selectivity to the pulse. Application to velocity selective arterial spin labeling is demonstrated.

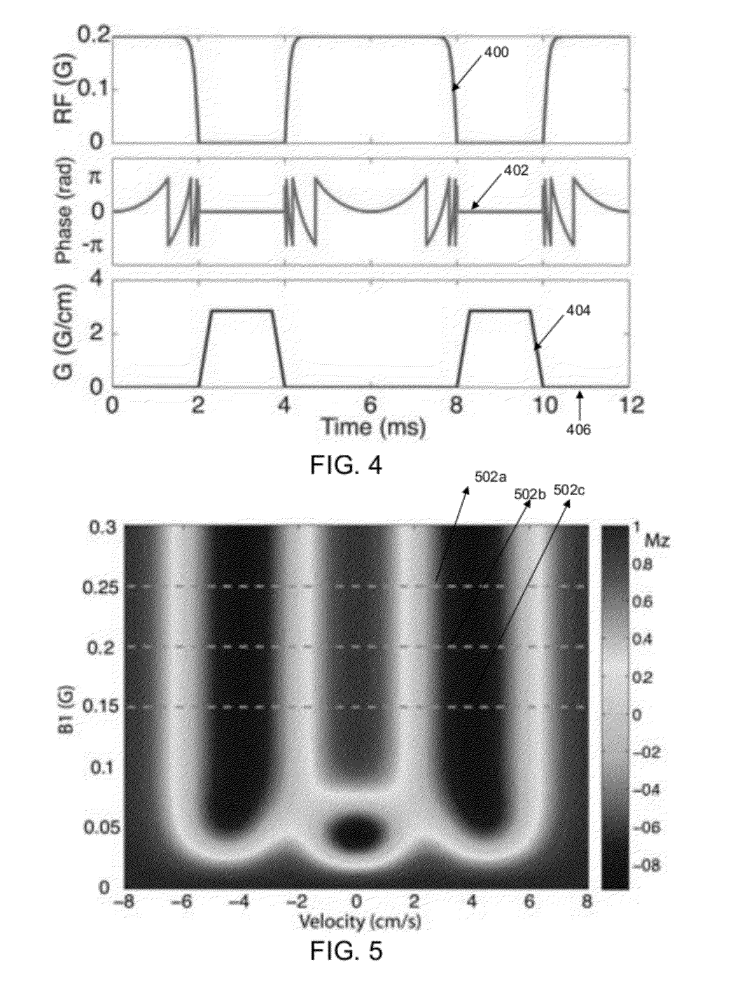

In some implementations, the BIR pulse can be modified to include two delays, during which equal gradient pulses 404 are applied (with curve 400 showing the corresponding RF field applied and curve 402 showing the phase of the applied pulse), as shown in FIG. 4. The horizontal axis 406 represents time. For the pulse shown, BIR-4 parameters were: wmax=39.8 KHz; zeta=15.2; tan(x)=63.6. The response of the pulse was simulated by numerical integration of the Bloch equations, including relaxation and constant flow in the direction of the gradient. T₁ and T₂ were assumed to be 1 s and 100 ms, respectively. The pulse was used in a velocity selective ASL experiment, with cutoff velocity 2 cm/s. The response was compared to that of a velocity selective pulse train using an adiabatic double spin echo with sech refocusing pulses and linear tip down/tip up pulses.

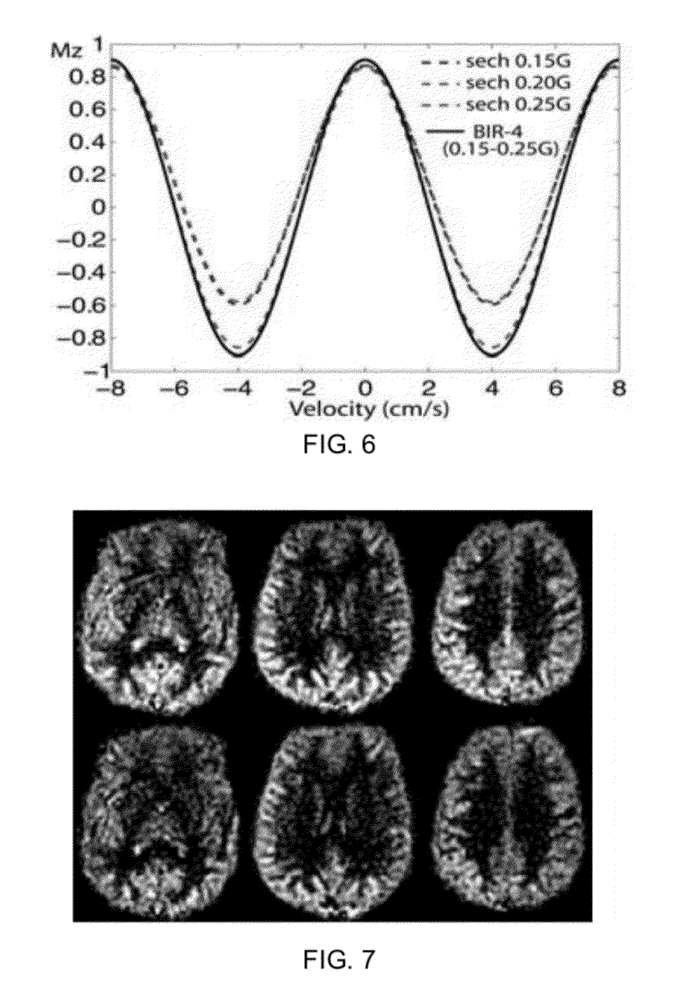

The calculated response of Mz to the pulse is shown in FIG. 2 as a function of B₁ and velocity, demonstrating B₁ insensitivity above a threshold and the expected cosinusoidal velocity dependence. For B₁=[0.15 0.20 0.25]G (dashed lines 502a, 502b and 502c in FIG. 5), Mz vs velocity is shown in FIG. 6 for both the BIR-4 and dual sech pulses. At B1=0.2 G where the linear pulses in the dual sech pulse train have the correct flip angle, the slightly higher amplitude of the cosine for the BIR-4 pulse is due to the shorter total pulse duration, resulting in less T₂ decay. At other values of B₁, the double sech is further degraded, while the amplitude of the response to the BIR-4 pulse is higher and indistinguishable across this B₁ range. In FIG. 7, the raw ASL signal is shown on the same scale for BIR-4 and double sech velocity selective labeling pulses. The average signal in the gray matter is 18% higher for the BIR-4 pulse.

Discussion

The BIR-4 based velocity sensitive pulse train was found to be superior to the double sech based pulse train in terms of T2 and B1 insensitivity. This is demonstrated using velocity selective ASL, but may also be useful for diffusion prepared pulse sequences or velocity based suppression of flowing spins. In one target applications, direct imaging of blood volume, the desired profile of M_{z} is proportional to sin(v) rather than cos(v), with the goal of suppressing static tissue. M_{z}<<sin(v) may also be useful for angiography.

This profile can be achieved using either pulse train with a 90° phase shift applied to the final sub-pulse. For this application, the advantage of the BIR-4 pulse is even more pronounced. For the cos(v) pulse, the double sech is accurately transparent at v=0 because the two sech pulses return magnetization to the same position as immediately following the tip down pulse, and the tip up pulse therefore returns magnetization accurately to the +Z axis, despite B₁ inhomogeneity. For the double sech sin(v) pulse train, the suppression at v=0 is proportional to cos²(a), where a is the nominal 90° linear pulse flip angle, while the BIR-4 based pulse train is inherently B₁ insensitive.

Useful Tangible Applications

The described techniques, apparatus and systems can be used for MR mapping of Oxygenation/OEF for identification of penumbra and monitoring of penumbra during and/or after treatment. Also, the described techniques, apparatus and systems can be used for MR mapping of Oxygenation/OEF for evaluation of tumors. Additionally, the described techniques, apparatus and systems can be used for MR mapping of Oxygenation/OEF for functional studies.

In another aspect, techniques, apparatus and systems are described for increasing the signal-to-noise ratio (SNR) and reduce imaging time.

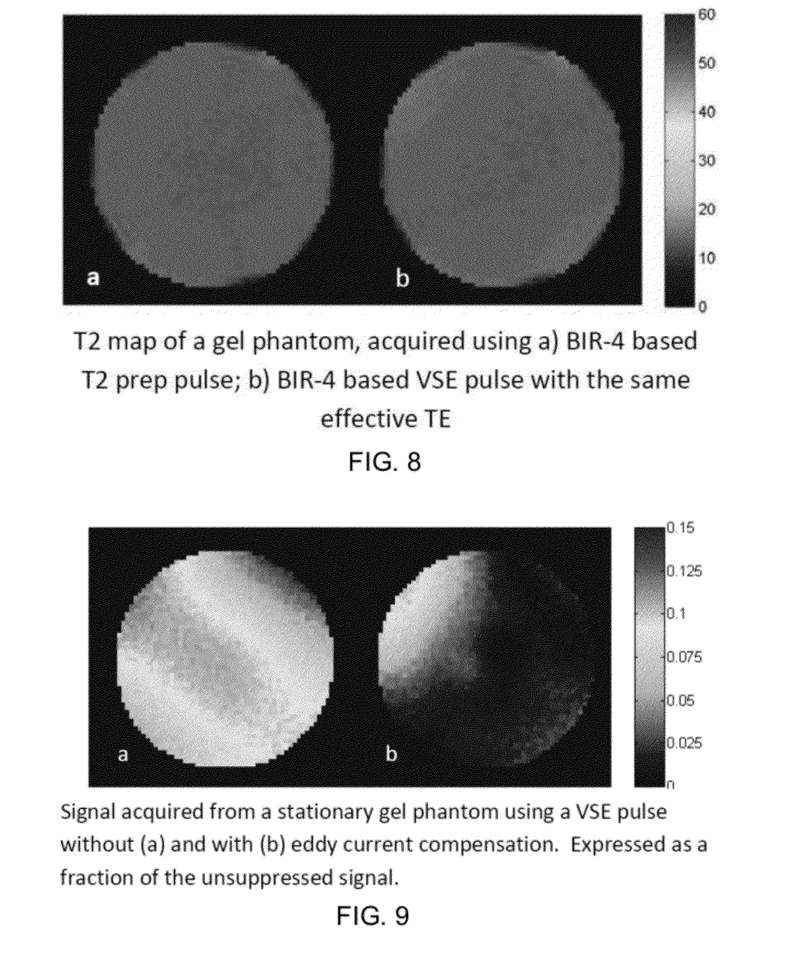

For example, the VSE (velocity selective excitation) pulse and T2 preparation pulses can be combined together into one module, with inserting adjustable time gaps in VSE pulse to generate desired T2 weighting for T2 estimation. Advantages of this technique can include: 1) lowering the minimum effective TE (echo time) used in T2 preparation; 2) eliminating the error from interaction between VSE and T2 preparation pulses; and 3) with less pulses used, the SAR (specific absorption rate) can reduced. FIG. 8 shows exemplary T2 measurement in a gel phantom using only a T2 preparation pulse. With this new combination method, the map can be very uniform.

Also, using eddy current compensation strategies, the noise caused by the static tissue signal can be reduced. FIG. 9 shows an exemplary signal acquired from a stationary gel phantom using a VSE pulse without (a) and with (b) eddy current compensation. Expressed as a fraction of the unsuppressed signal.

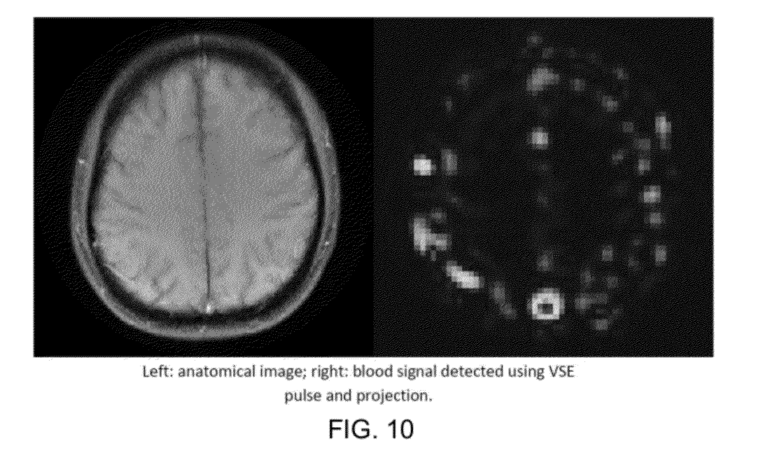

With adjustment of the imaging parameters in the VSE pulse, the sensitivity can be changed to the size of the vessels which are of interests. FIG. 10 shows an example provided by using higher cutoff velocity of 3 cm/s instead of the original 2 cm/s; the signal from the blood is clearly matched with the vessel structures, and an effective suppression of static tissue signal is observed.

With the same principle, the oxygenation of arterial blood can also be measured using VSE pulse. Using additional arterial nulling pulses, venous and arterial contributions to the signal can be separated, and the oxygenation level of each compartment measured by T2 measurement.

Imaging of Oxygen Extraction Fraction Using Velocity Selective Excitation with Arterial Nulling (VSEAN)

Measuring metabolic rate of a tissue benefits our understanding its physiology and functions. In the brain, OEF provides information on the viability of brain tissue, is thought to be a more specific indicator of ischemic penumbra in stroke than contrast based perfusion MRI. Functional studies can demand better measure of the Cerebral Metabolic Rate of Oxygen (CMRO2), which can be calculated via CMRO2=OEF*CBF.

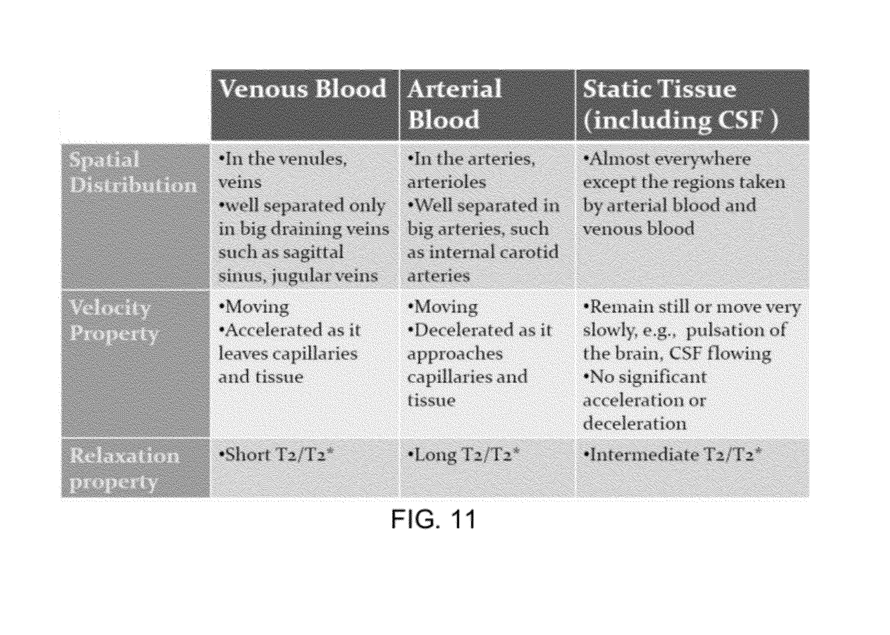

To target only the venous blood, the venous blood, arterial blood and static tissue (including CSF) can be separated. FIG. 11 shows characteristics of separate venous blood, arterial blood and static tissue (including CSF).

Also, to target only the venous blood, various non-invasive MRI techniques can be implemented. For example, TRUST can be used to separate venous blood based on its spatial distribution—acquire signal in big veins (e.g., sagittal sinus). TRUST can provide pure venous signal, no arterial blood signal, and no tissue signal. Also, TRUST can provide high signal intensity. TRUST also provides only one oxygenation value for the whole brain, with no spatial information. Moreover, TRUST images far from the tissue of interest.

BOLD effect is a non-invasive MRI method that measures R2/R2′ and use model to extract Oxygenation information. The BOLD effect can provide signal acquisition localized to tissue, spatial mapping possible. The signal in the BOLD effect comes from all the sources—venous blood, arterial blood, static tissue. In addition, the BOLD effect uses a complicated model.

QUIXOTIC is another non-invasive MRI technique that can be used to separate venous/arterial blood based on their acceleration/deceleration properties. The static tissue signal can be subtracted out. In QUIXOTIC, the signal acquisition can be localized to tissue, and spatial mapping is possible. Also, in QUIXOTIC, the signal from arterial blood and static tissue can be under control. However, in QUIXOTIC, the diffusion effects (from CSF) can cause subtraction errors and may contaminate venous signal. Also, subtraction in QUIXOTIC ca generate one data point every two TR, low time efficiency. Moreover, an inversion pulse in QUIXOTIC to suppress arterial signal can reduce venous signal.

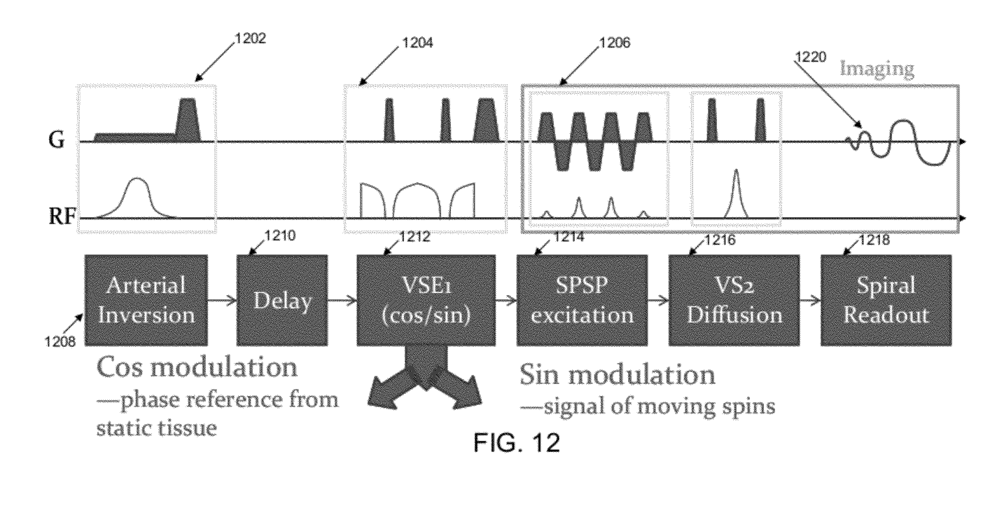

The VSEAN technique disclosed in this document can invert upstream arterial blood and acquire images at its null point to eliminate arterial signal. Also, Velocity Selective Excitation combined with flowing signal projection can efficiently suppress static tissue signal. FIGS. 12-15 show an exemplary process and associated signals for implementing VSEAN.

With respect to FIG. 12, 1202 represents a gradient field and a corresponding RF pulse that may be used to excite arterial blood. In some implementations, the pulses 1202, 1204, 1206 and 1220 may be similar to the pulses 202, 204, 206 and 208 previously discussed. After arterial blood is magnetized, it undergoes a natural relaxation process. During the relaxation process, the magnetic field of the arterial blood undergoes arterial inversion (box 1208). After certain time period during the relaxation, the magnetic field goes through zero value while changing the polarity of magnetization during relaxation. The delay 1210 may be adjusted such that the rest of the sequence of steps described next can occur when the arterial magnetization is at a zero magnitude. In other words, the contribution of the arterial portion can be excluded.

Block 1212 represents the application of the first velocity-selective pulse, previously described, with the corresponding pulse shapes shown in 1204. The first velocity-selective pulse causes a selective excitation that creates a sine dependence. Next, pulses 1206 may be applied (Spatial Spectral, or SP, excitation 1214). This step may be followed by a second pulse in the second velocity selective diffusion box 1216, which then creates a sin-squared dependency of the magnetic field with the velocity. A spinal readout 1218 (also shown in waveform 1220) may then be performed to obtain an image of the subject.

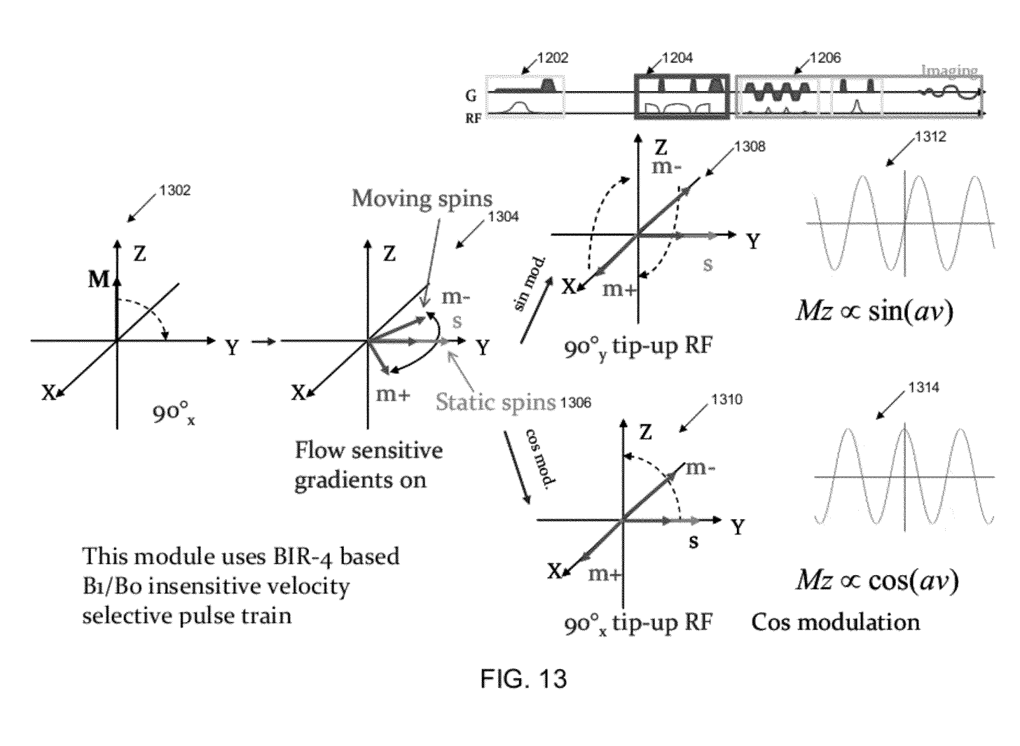

FIG. 13 shows relationship between magnetization spin directions and the effect of applying velocity-selective excitation pulses. With respect to graph 1302, a magnetic field applied in the Z direction of the XYZ coordinate space is shown. The moving spins m+ and m− (1304) are in the XY plane and the static spins 1306 are along the Y coordinate. With sin modulation, the resulting spin rotation is a 90 degree y tip-up (1308) of the spins and with cos modulation, the resulting spin rotation is a 90 degree x tip-up (1310). The sequence of pulses 1202, 1204, 1206 thus creates sin dependency (1312) and cos dependency (1314) of magnetic excitation field on the velocity.

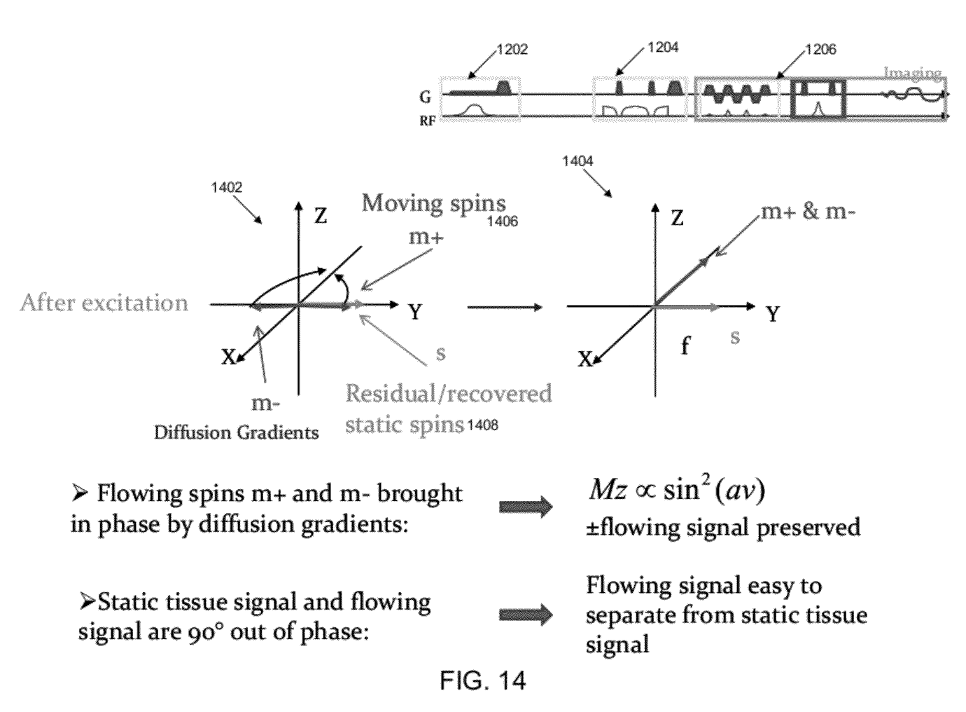

FIG. 14 shows the effect of velocity-selective excitation pulses on magnetization spins caused in a subject. As depicted in 1402, after excitation with pulses 1202, 1204, 1206, moving spins 1406 are brought in phase by the diffusion gradients, thereby creating a sin-squared dependency. With reference to 1404, 1406, the static tissue signal and the flow signals are 90 degrees out of phase, thereby allowing easy separation of the flow signals from the static signals.

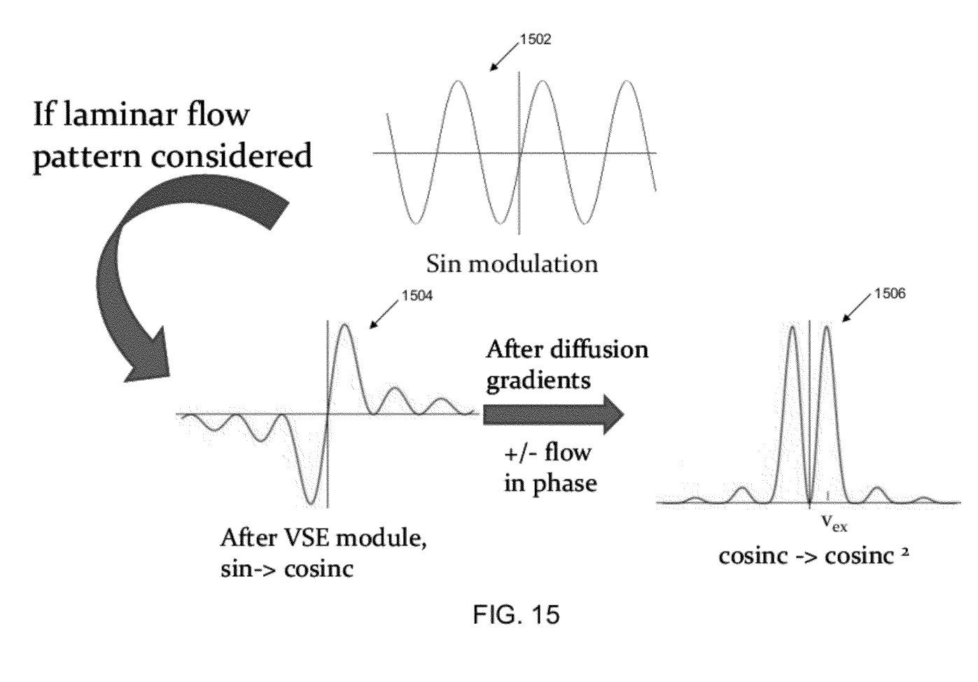

FIG. 15 show an exemplary process and associated signals for implementing VSEAN. Under the assumption that the flow is laminar (which is typically the case for blood flow through blood vessels), a sin modulated function 1502 is converted into a cosinc function 1504 after the application of velocity-selective excitation, which then is converted into a cosinc-squared function 1506 after diffusion gradients.

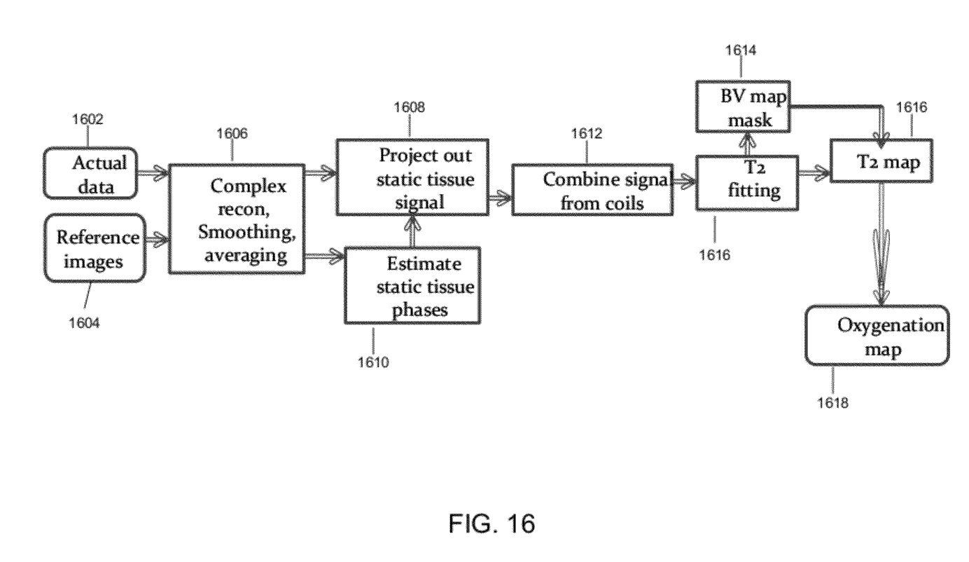

FIG. 16 shows a block diagram of an exemplary data processing flow. Actual data 1602 and reference images 1604 may be processed through a complex reconstruction, smoothing and averaging operation 1606. The static tissue phases in the collected data may be measured (1610) and projected out (1608) of the measured data. The signals obtained from each coils may be combined (1612), a T2 fitting operation may be performed (1616) to obtain a blood volume (BV) map. Based on these, a T2 map may be obtained (1620). Using the previously generated map data, an oxygenation map may be obtained (e.g., venous oxygenation map) at 1618.

VSEAN data acquisition can include acquiring images with cos modulation—static tissue phase reference, and acquiring images with sin modulation—actual data. Imaging parameters can include single slice spin echo with spiral-out readout, spatial spectral excitation, vex=2 cm/s, z-direction. FIG. 16 shows a block diagram of an exemplary data processing technique.

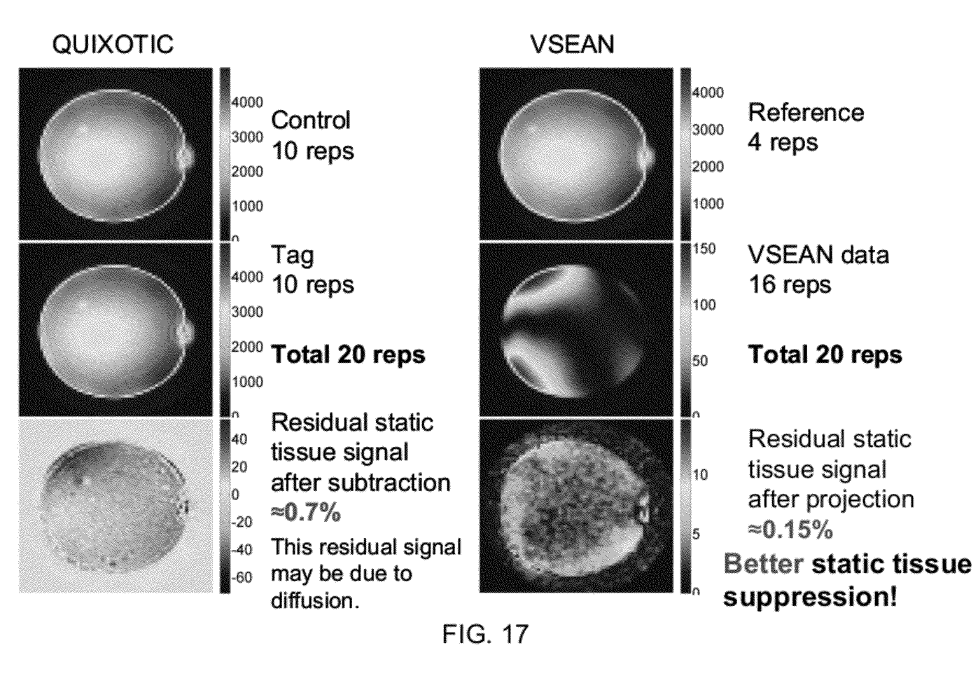

FIG. 17 shows tissue suppression on a silicone phantom. As can be seen, the use of VSEAN techniques results in better suppression of signals from static tissue, an almost 0.7/0.15 or a five-fold improvement over previous techniques such as QUIXOTIC.

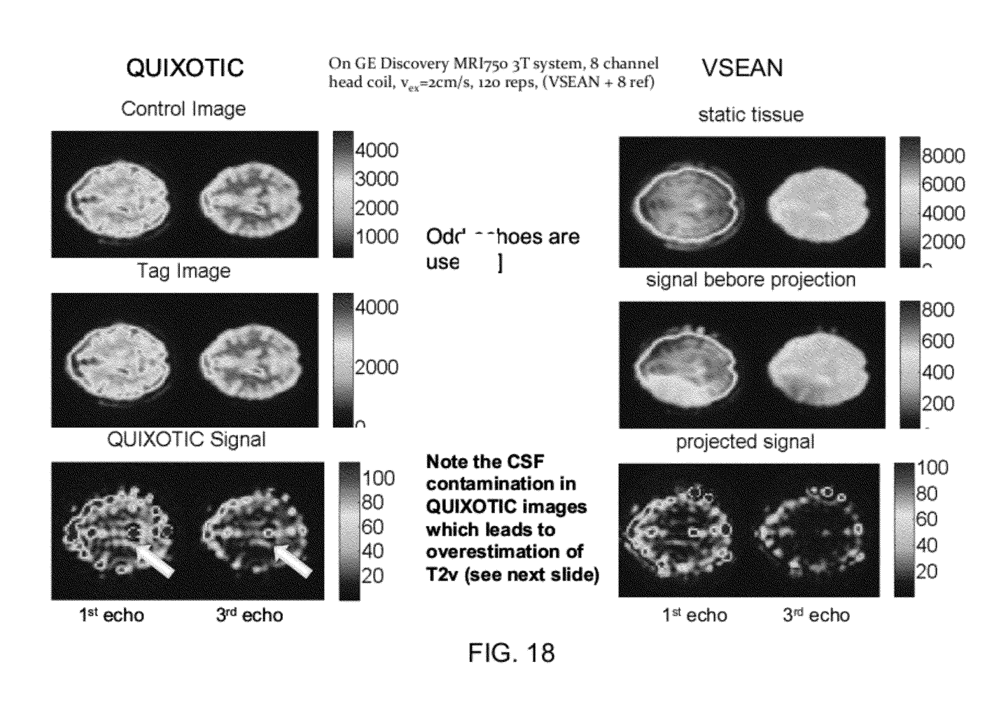

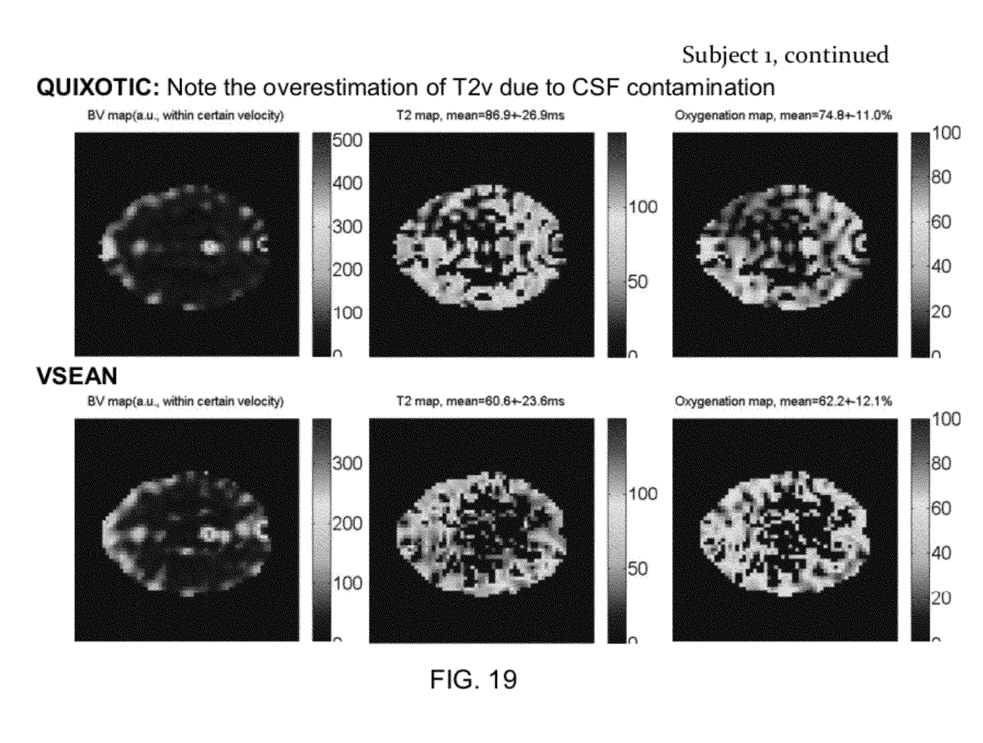

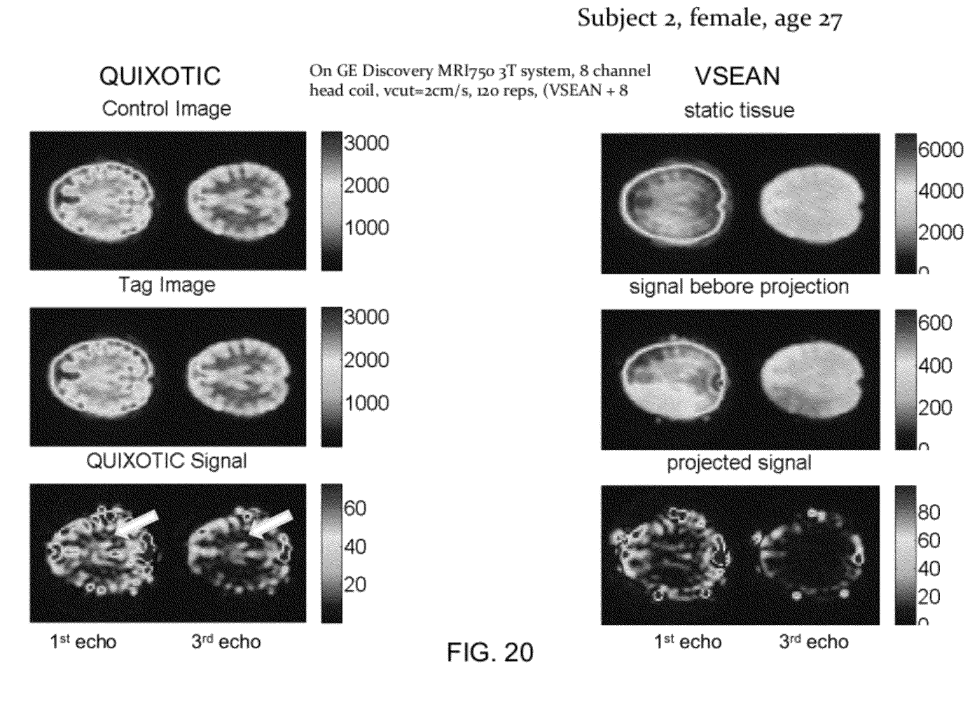

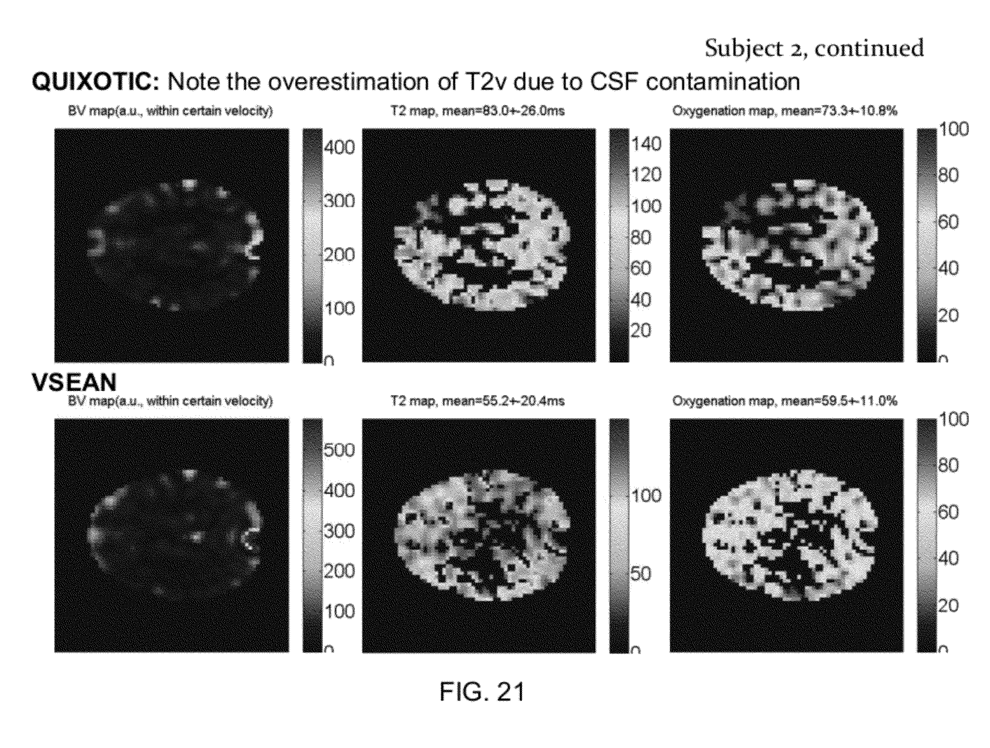

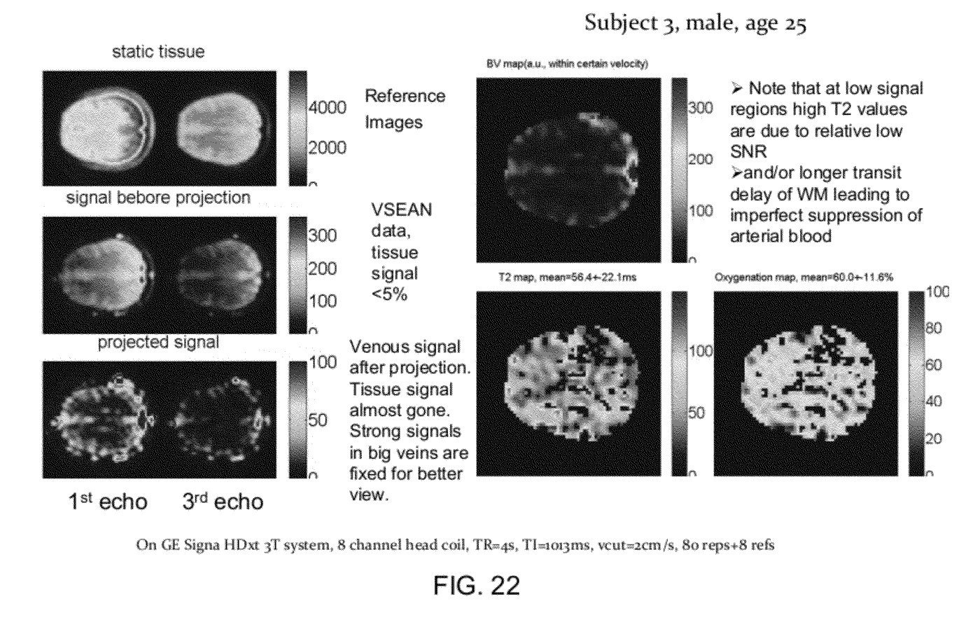

FIGS. 18-22 show comparisons of VSEAN and QUIXOTIC.

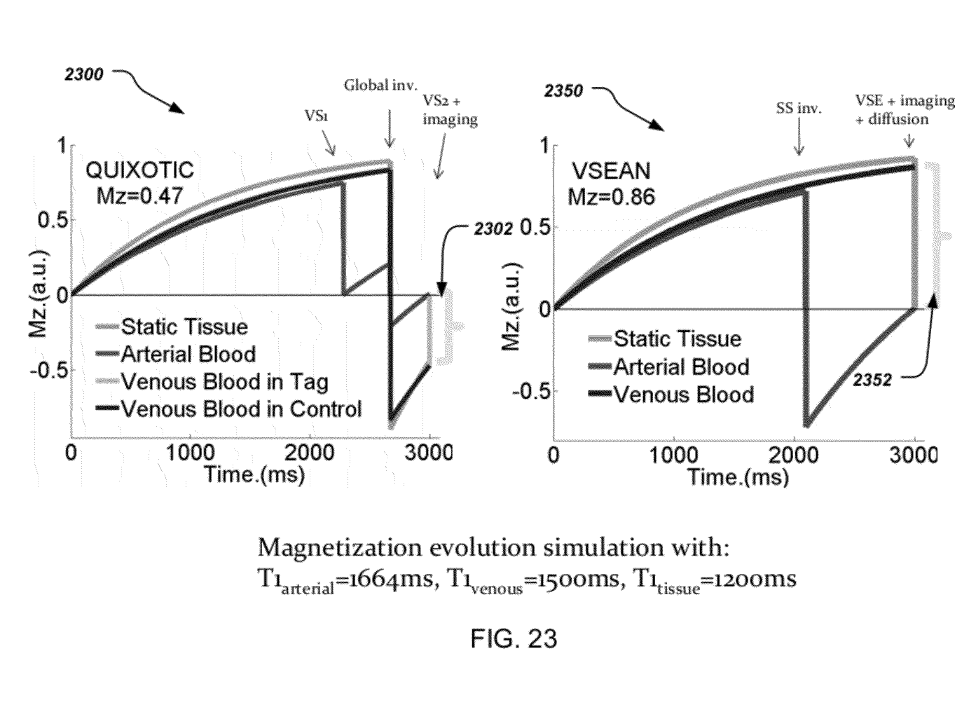

FIG. 23 shows a comparison of the venous signal between VSEAN and QUIXOTIC. The charts 2300 and 2350 were previously discussed with respect to FIGS. 1A and 1B. The improvement in Mz (magnetization in the z direction) of observation is evident from the charts, with the value increasing from approximately 0.47 units in the case of QUIXOTIC to 0.86 units in the case of VSEAN.

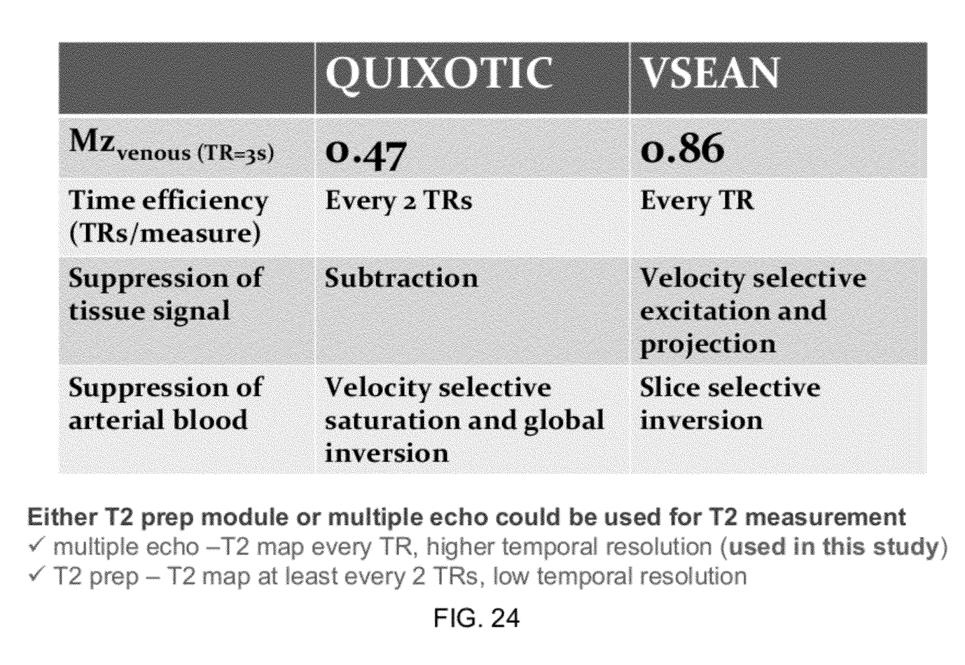

FIG. 24 is a table shows a summary of comparison between VSEAN and QUIXOTIC.

Compared to QUIXOTIC, VSEAN can provide higher venous blood signal and can double the time efficiency by using VSE instead of subtraction. Also, VSEAN can provide better tissue (CSF) suppression than QUIXOTIC, and be less sensitive to small motion, physiologic noise than QUIXOTIC. Also, VSEAN can provide for multi-echo acquisition that allows faster T2 mapping than T2-preparation based method, and in VSEAN it is possible to observe brain activities. Transit delay in VSEAN, which can affect the nulling of arterial blood could be solved by velocity selective inversion.

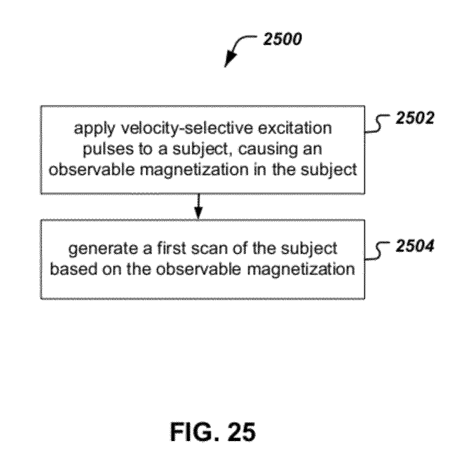

FIG. 25 is a flow chart representation of an MRI process 2500. At 2502, velocity-sensitive excitation pulses are applied to a subject. The velocity-selective excitation pulses may have the previously discussed mathematical properties such as zero value at zero velocity, zero slope at zero velocity, and so on. The application of the pulses causes an observable magnetization in the subject. The observable magnetization generally corresponds to the transverse magnetization in the subject. At 504, a first scan is generated of the subject using the observable magnetization. In some implementations, the first scan may be generated based on data captured from the subject. As previously discussed, the velocity-selective excitation pulses may include velocity-selective excitation pulses including arterial nulling.

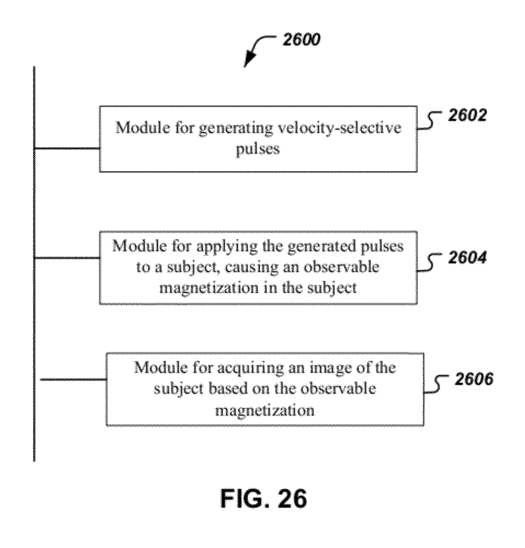

FIG. 26 is a block diagram representation of an apparatus 2600 for MRI. The module 2602 is for generating velocity-selective pulses. The techniques used for generating velocity-selective pulses are disclosed, e.g., with respect to FIGS. 12-16 in the present document. The module 2604 is for applying the generated velocity-selective excitation pulses to a subject. For example, in some implementations, multiple coils may be used to apply the excitation pulses to the subject. The module 2606 is for acquiring an image of the subject based on the observable magnetization. As previously discussed, multiple spin echo acquisition may be used. In some implementations, a spinal read out with matrix size 32 may be used. The modules 2602, 2604 and 2606 may further be configured to implement one or more techniques described in this document.

It will be appreciated that new and novel techniques for magnetic resonance imaging of a subject are disclosed in this document. In one aspect, the MRI images are obtained by applying velocity-selective excitation pulses to the subject. In one aspect, the velocity-selective pulses are designed so that the contribution to the observable magnetization field by zero velocity (or static) portion of the subject is suppressed or completely eliminated. In some embodiments, arterial nulling pulses are used along with velocity-selective excitation pulses, so that the contribution to the observable magnetization of the subject by arterial blood is suppressed or eliminated.

It will further be appreciated that the techniques disclosed in this document find applications in a variety of medical areas such as magnetic resonance imaging and magnetic resonance angiography. In one advantageous aspect, the suppression of static portion contribution (e.g., body tissue) and arterial blood portion enables obtaining high signal to noise ratio venous oxygenation maps, which are useful in several media diagnostic applications.

Implementations of the subject matter and the functional operations described in this specification can be implemented in various MRI machines and digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non volatile memory, media and memory devices, including by way of example semiconductor memory devices, e.g., EPROM, EEPROM, and flash memory devices. The processor and the memory can be supplemented by, or incorporated in, special purpose logic circuitry.

While this specification contains many specifics, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments.

Only a few implementations and examples are described and other implementations, enhancements and variations can be made based on what is described and illustrated in this application.

What is claimed is:

1. A magnetic resonance imaging method, comprising:
applying velocity-selective excitation pulses to a subject, causing an observable magnetization in the subject; and
generating a first scan of the subject based on the observable magnetization.

2. The method of claim 1, wherein the velocity-selective excitation pulses are generated to facilitate imaging of a moving magnetization portion of the subject only.

3. The method of claim 1, wherein the subject comprises an arterial blood portion and a venous blood portion and wherein the velocity-selective excitation pulses include velocity-selective excitation and arterial nulling (VSEAN) pulses such that the observable magnetization excludes a contribution from the arterial blood portion.

4. The method of claim 3, further comprising:
applying the VSEAN pulses to generate a second scan of the subject;
mapping arterial T2 and oxygenation by subtracting the second scan from the first scan of the subject.

5. The method of claim 1, wherein the velocity-selective excitation pulses include velocity-selective excitation and arterial nulling (VSEAN) pulses, the method further comprising:
applying a combination of VSEAN and multi-echo T2 measurements to map venous T2 and oxygenation of the subject.

6. The method of claim 1, wherein the subject comprises an arterial blood portion and a venous blood portion and wherein the velocity-selective excitation pulses include velocity-selective excitation and arterial nulling (VSEAN) pulses, the method further comprising:
applying a combination of VSEAN and T2 preparation to the subject to enable mapping of venous T2 and oxygenation.

7. The method of claim 1, wherein the velocity-selective excitation pulses are designed to produce the observable magnetization that is a positive function of a subject velocity, so that signals from spins and velocities of mixed sign do not cancel each other.

8. The method of claim 1, in which the velocity-selective excitation pulses comprise pulses that produce the observable magnetization of the subject as a function of velocity of a moving portion of the subject, such that the function has a zero slope at zero velocity, thereby suppressing signals generated from a static portion of the subject signal.

9. The method of claim 8, wherein the function is proportional to sine-square of v, wherein v is the velocity of the moving portion.

10. The method of claim 1, wherein the operation of generating the first scan comprises:
using phase sensitive detection to generate the first scan such that contribution from a static tissue portion of the subject is suppressed.

11. The method of claim 10, where the operation of using phase sensitive detection comprises:
projecting phase data onto a phase reference.

12. A magnetic resonance imaging apparatus, comprising:
a generator that generates velocity-selective excitation pulses;
a plurality of coils that applies the generated pulses to a subject; and
a data processor that reconstructs an image of the subject by combining data from the plurality of coils.

13. The apparatus of claim 12, wherein the velocity-selective excitation pulses are generated to facilitate imaging of a moving magnetization portion of the subject only.

14. The apparatus of claim 12, wherein the subject comprises an arterial blood portion and a venous blood portion and wherein the velocity-selective excitation pulses include velocity-selective excitation and arterial nulling (VSEAN) pulses such that the observable magnetization excludes a contribution from the arterial blood portion.

15. The apparatus of claim 14, wherein the plurality of coils further applies the VSEAN pulses to generate a second image of the subject; and
a mapper maps arterial T2 and oxygenation by subtracting the second image from the first image of the subject.

16. The apparatus of claim 12, wherein the velocity-selective excitation pulses include velocity-selective excitation and arterial nulling (VSEAN) pulses, and wherein the plurality of coils is adapted to apply a combination of VSEAN and multi-echo T2 measurements to map venous T2 and oxygenation of the subject.

17. The apparatus of claim 12, wherein the subject comprises an arterial blood portion and a venous blood portion and wherein the velocity-selective excitation pulses include velocity-selective excitation and arterial nulling (VSEAN) pulses, wherein the plurality of coils is further adapted to apply a combination of VSEAN and T2 preparation to the subject to enable mapping of venous T2 and oxygenation.

18. The apparatus of claim 12, wherein the velocity-selective excitation pulses are designed to produce the observable magnetization that is a positive function of a subject velocity, so that signals from spins and velocities of mixed sign do not cancel each other.

19. The apparatus of claim 12, in which the velocity-selective excitation pulses comprise pulses that produce the observable magnetization of the subject as a function of velocity of a moving portion of the subject, such that the function has a zero slope at zero velocity, thereby suppressing signals generated from a static portion of the subject signal.

20. The apparatus of claim 19, wherein the function is proportional to sine-square of v, wherein v is the velocity of the moving portion.

21. The apparatus of claim 12, wherein the operation of generating the first scan comprises:
using phase sensitive detection to generate the first scan such that contribution from a static tissue portion of the subject is suppressed.

22. The apparatus of claim 21, where the operation of using phase sensitive detection comprises:
projecting phase data onto a phase reference.

23. A magnetic resonance imaging apparatus, comprising:
means for generating velocity-selective pulses;
means for applying the generated pulses to a subject, causing an observable magnetization in the subject; and
means for acquiring an image of the subject based on the observable magnetization.
